(19) United States
(12) Patent Application Publication
(10) Pub. No.: US 2003/0140818 A1
(43) Pub. Date: Jul. 31, 2003

(54) Production of polysaccharide

(75) Inventor: Thomas J. Pollock, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignees: Shin-Etsu Bio, Inc.; Shin-Etsu Chemical Co., Ltd.

(21) Appl. No.: 09/815,207

(22) Filed: Mar. 22, 2001

Related U.S. Application Data
(63) Continuation-in-part of application No. 09/607,248, filed on Jun. 30, 2000.
(60) Provisional application No. 60/142,121, filed on Jul. 2, 1999.

(51) Int. Cl. C08L 5/00; C12P 19/04; C12N 1/20; C08B 37/00

(52) U.S. Cl. 106/162.1; 106/205.01; 435/101; 435/252.3; 536/123

(57) ABSTRACT

The present invention provides for a polysaccharide produced by subjecting a Sphingomonas bacterium modified with a S7c6 gene cluster or segment thereof to aerobic fermentation in a nutrient aqueous broth for a time sufficient to produce the polysaccharide dissolved therein, including L-Rhap, D-Glcp and 2-deoxy-β-D-arabino-HexpA in a molar ratio of 1:3:1, wherein the polysaccharide has at least 20% less glucose per repeat unit compared to a heteropolysaccharide S-7 produced by an unmodified Sphingomonas strain S7, and the segment includes at least the spsB and rhsACBD genes.

### CROSS REFERENCE TO RELATED APPLICATIONS

This application is a continuation-in-part application of U.S. application Ser. No. 09/607,248, filed Jun. 30, 2000, which claims the benefit of priority of U.S. Provisional Application No. 60/142,121, filed on Jul. 2, 1999.

### BACKGROUND OF THE INVENTION

The present invention relates to a polysaccharide which is produced by the fermentation of a nutrient medium with a particular species of bacteria. Specifically, the present invention relates to an extracellular polysaccharide produced from fermentation of a modified bacterium of Sphingomonas strain S7.

Sphingomonas strain S7 was recently reassigned to a new genus. See T. J. Pollock, 1993, Journal of General Microbiology, volume 139, pages 1939-1945. An unmodified Sphingomonas strain S7 produces polysaccharide S-7 (hereinafter referred to as “EPS S-7” or “S-7”) which is the subject of four expired patents. (1) U.S. Pat. No. 3,960,832 issued to Kang et al. on Jun. 1, 1976 which discloses a single composition of matter; (2) U.S. Pat. No. 3,915,800 issued to Kang et al. on Oct. 28, 1975 which discloses the growth of a naturally occurring bacterial strain Azotobacter indicus (deposited as ATCC 21423) in a submerged aerated culture in a nutrient medium and the recovery of the polysaccharide; (3) U.S. Pat. No. 3,894,976 issued to Kang et al. on Jul. 15, 1975 which discloses the use of S-7 in water based paints; and (4) U.S. Pat. No. 3,979,303 issued to Kang et al. on Sep. 7, 1976 which discloses the use of S-7 in oil well drilling. In addition, U.S. Pat. No. 5,772,912 issued to Lockyer et al. on Jun. 30, 1998 discloses the use of S-7 in anti-icing formulations and U.S. Pat. No. 4,462,836 issued to Baker et al. on Jul. 31, 1984 discloses the use of S-7 in cement.

Furthermore, published literature concerning this polysaccharide is limited to a 1977 review by the inventors of the Kang et al. patents which is based on the information in their published patents (See Kang et al., “A New Bacterial Heteropolysaccharide In Extracellular Microbial Polysaccharides,” American Chemical Society, pp. 220-230 (1977)), and two brief studies by others concerning conditions for growing the naturally occurring bacterium (See Lee, et al., “Compositional Consistency of a Heteropolysaccharide-7 Produced by Beijerinckia indica,” Biotechnology Letters, 19 (1997) and Naumov et al., “Optimal Nitrogen and Phosphorous Concentrations in the Growth Medium for Exopolysaccharide Biosynthesis by Beijerinckia indica,” Mikrobiologiya, pp. 856-857 (1985)).

Polysaccharides like S-7 have several applications, for example, as a thickener, suspending agent and stabilizer. In addition, S-7 can be used to modify the viscosity of aqueous solutions. Although S-7 has several applications, it is one of the purposes of the present invention to provide polysaccharides with improved characteristics.

### SUMMARY OF THE INVENTION

Accordingly, a novel polysaccharide (hereinafter sometimes referred to as “EPS S7c6” or “S7c6”) has been found with increased viscosity at lower concentrations. The preparation of the polysaccharide includes constructing modified derivatives of the naturally occurring parental bacterium Sphingomonas strain S7 by genetic engineering. The modified derivatives exhibit increased conversion of the carbon source in a nutrient culture medium into the product exopolysaccharide S-7, compared to the unmodified parent strain.

A polysaccharide was prepared from one of the genetically-modified derivatives that has a carbohydrate composition which is different from the parent polysaccharide S-7, and which has improved viscosity characteristics compared to polysaccharide S-7. Specifically, the polysaccharide was produced by subjecting a Sphingomonas bacterium modified with a S7c6 gene cluster or segment thereof to aerobic fermentation in a nutrient aqueous broth for a time sufficient to produce the polysaccharide dissolved therein. The segment includes at least the spsB and rhsACBD genes and the polysaccharide includes L-Rhap, D-Glcp and 2-deoxy-β-D-arabino-HexpA in a molar ratio of approximately 1:3:1, respectively. The polysaccharide includes predominantly the following pentasaccharide repeating unit:

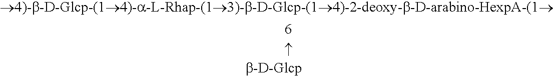

Furthermore, the polysaccharide has at least 20% less glucose per repeat unit, preferably at least 25% less glucose per repeat unit, compared to a heteropolysaccharide S-7 produced by an unmodified Sphingomonas strain S7.

The present invention also provides for a method for increasing the viscosity of an aqueous solution. The method includes adding to an aqueous solution a viscosity increasing effective amount of a polysaccharide which includes L-Rhap, D-Glcp and 2-deoxy-β-D-arabino-HexpA in a molar ratio of 1:3:1. The polysaccharide used in this method is produced by subjecting a Sphingomonas bacterium modified with a S7c6 gene cluster or segment thereof to aerobic fermentation in a nutrient aqueous broth for a time sufficient to produce the polysaccharide dissolved therein, and the segment includes at least the spsB and rhsACBD genes. Furthermore, it is preferable that the polysaccharide has at least 20% less glucose per repeat unit compared to a heteropolysaccharide S-7 produced by an unmodified Sphingomonas strain S7 and even further preferable that the polysaccharide has at least 25% less glucose per repeat unit compared to a heteropolysaccharide S-7.

The present invention also provides for a fermentation broth obtained by subjecting a Sphingomonas bacterium modified with a S7c6 gene cluster or segment thereof to aerobic fermentation in a nutrient aqueous broth for a time sufficient to produce a dissolved polysaccharide. The segment includes at least the spsB and rhsACBD genes. The polysaccharide includes L-Rhap, D-Glcp and 2-deoxy-β-D-arabino-HexpA in a molar ratio of 1:3:1 and has at least 20% less glucose per repeat unit compared to a heteropolysaccharide S-7 produced by an unmodified Sphingomonas strain S7. It is preferable that the polysaccharide has at least 25% less glucose per repeat unit compared to the heteropolysaccharide S-7.

### BRIEF DESCRIPTION OF THE DRAWINGS

FIG. 1 shows the organization of the sps gene cluster of Sphingomonas strain S7.

FIG. 2 shows the DNA sequence of the S7c6 cloned segment.

FIG. 3 shows the DNA sequence of the pgm gene of Sphingomonas strain S7.

FIG. 4 shows the amino acid sequence of the pgm protein of Sphingomonas strain S7.

FIG. 5 shows the MALDI-TOF mass spectrum of the products generated by per-O-deuteriomethylation of EPS S7c6.

FIG. 6A shows the ID ¹H NMR spectrum of per-O-deuteriomethylated EPS S7c6.

FIG. 6B shows the ID ¹H NMR spectrum (anomeric region) of per-O-deuteriomethylated EPS S7c6.

FIG. 7A shows the ¹H-¹³C HSQC spectrum (anomeric region) of per-O-deuteriomethylated EPS S7c6.

FIG. 7B shows the ¹H-¹³C HSQC spectrum (ring carbons) of per-O-deuteriomethylated EPS S7c6.

### DETAILED DESCRIPTION OF THE INVENTION

As stated above, polysaccharides like S-7 can be used to modify the viscosity of aqueous solutions. Several polymers have this capacity, such as xanthan gum, cellulose, and guar. A new polymer like that produced by Sphingomonas strain S7 containing plasmid pRK-S7c6, which is described below in the Example and which represents a new composition of matter, shows increased viscosity at lower concentrations.

The following example is provided to assist in further understanding the present invention. The particular materials and conditions employed are intended to be further illustrative of the invention and are not limiting upon the reasonable scope thereof.

### EXAMPLE

### Culture Conditions

A culture medium for Sphingomonas strain S7 and the derivatives was prepared including the following components dissolved in 1 liter of tap water: 20 g glucose, 1 g ammonium nitrate, 0.5 g soluble soy protein, 3.2 g dipotassium phosphate, 1.6 g monopotassium phosphate, 0.2 g magnesium sulfate, and 0.1% v/v of concentrated trace minerals. The concentrated trace minerals were dissolved in deionized water at the following final concentrations: 10 mM FeCl₃, 10 mM ZnCl₂, 10 mM MnCl₂, 1 mM CoCl₂, 1 mM Na₂MoO₄, and 1 mM CuSO₄. For a solid medium, agar was added to 1.5% v/v before sterilization by autoclaving at 121° C. for 20 min. Bacteria cultured on agar plates were then incubated at 30° C. for 2-4 days. For culture volumes of 10-500 mL, bacteria were grown in liquid medium at 30° C. in baffled Erlenmeyer flasks with rotary shaking at 160 rpm. All culture volumes were not more than one-half of the maximum flask capacity.

Seed cultures for the fermentations were prepared in two stages. First, a single representative colony was inoculated into 100 mL of liquid medium containing selective antibiotics as required and grown for 18 hours until mid to late exponential phase, and then dispensed into 2 mL aliquots in plastic tubes and frozen at −70° C. Second, to prepare a 5% v/v seed culture for a 4 L fermentation, one frozen tube was thawed and a portion, usually 0.5-1.5 mL, was inoculated into 250 mL of medium and shaken for 18 hours. After this period, the seed cultures usually achieved an optical density at 600 nm of 3-6, with a final pH of 5.5-6.5.

Fermentations were carried out in 3 to 4 liters of medium using New Brunswick BioFlo III and 3000 equipment. The round bottomed vessel had a marine impeller at the top pushing downward and two equally spaced 6-bladed Rushton impellers at the midpoint and at the bottom of the shaft. No baffles were present on the periphery of the vessel. Agitation was initially 50 rpm and was under the control of the dissolved oxygen sensor which was set to a minimum of 20-30%. Agitation increased as the culture became dense to a maximum of 1000 rpm. Air was supplied at 1 volume per minute. The culture pH was initially adjusted to 7.0. During the exponential phase of growth, it decreased naturally to about 6.0-6.2, then after the ammonium was depleted, the pH increased to around 6.5-6.8, and then decreased slowly to the end of the cycle to around 5.8-6.2. Control of pH with the addition of KOH or HCl was not necessary. Small amounts of antifoam (1-5 mL, Sigma 204) were added as needed during the exponential phase of growth. As the culture viscosity increased above 10,000 cp (Brookfield LVTDV-II, spindle 4, 12 rpm, 25° C.), the dissolved oxygen decreased to zero, the temperature which was initially set to 30.0° C., began to fluctuate by 0.3° C., and as much as one-half of the broth volume, the portion furthest from the impellers, remained stationary. For each fermentation, an automatic record was kept of temperature, dissolved oxygen, pH, and agitation. Measurements were made for the absorbance at 600 nm, ammonium concentration, residual glucose concentration, viscosity and dry weight of the biomass precipitated with two volumes of isopropyl alcohol.

### Genetic Modifications

Preparation of a Library of S7 Genes

Sphingomonas strain S7 was cultured in 5 mL of YM medium by shaking at 30° C. After adding 0.55 mL of 10×TE (100 mM Tris-HCl,10 mM EDTA, pH 8), 0.3 mL of 10% sodium dodecylsulfate and 0.03 mL of 20mg/mL proteinase K, the cultures were incubated with shaking for one hour at 65° C. After adding 1 mL of 5M NaCl and 0.8 mL of 10% CTAB (hexadecyltrimethylammoniumbromide) in 1M NaCl, the lysates were incubated for 30 minutes at 65° C. and then extracted once with chloroform and once with phenol:chloroform (1:1). The upper aqueous phase was removed and added to a 0.6 volume of isopropyl alcohol and then dried. The precipitate was resuspended with a mixture of 0.6 mL of 1×TE containing 0.7 M NaCl and 0.1 mL of 10% CTAB in 1M NaCl, incubated 30 min at 65° C., extracted once with chloroform, and then precipitated with two volumes of ethanol. After drying, the pellet was resuspended in 0.1 mL of 1×TE. High molecular weight DNA was partially digested with SalI enzyme. The SalI-digested S7 DNA was treated with Klenow DNA polymerase to add dCMP and dTMP to the cohesive ends, heated for 20 min at 70° C. and then precipitated with ethanol. The vector plasmid pRK311 was digested with BamHI enzyme, purified by phenol extraction and ethanol precipitation, treated with Klenow DNA polymerase to add dGMP and dAMP, and purified. Equal molar amounts of vector and insert fragments were ligated (T4 DNA ligase), packaged into bacteriophage λ (Gigapack IIXL; Stratagene) and transfected into Escherichia coli DH5α. One library of 1,700 and another of 3,400 tetracycline-resistant (Tet^{r}) colonies were separately pooled and frozen. The Tet^{r} colonies (10 of 10 tested) contained inserts of 25 to 30 kbp with internal SalI restriction sites.

Isolation of the S7c6 Gene Cluster

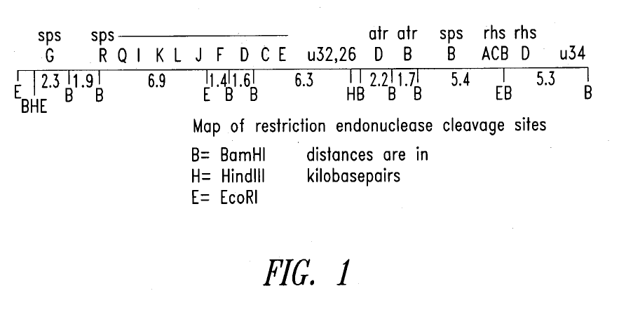

Cells representing the entire pooled library were mixed with cells of an exopolysaccharide (eps)-negative mutant (such as S88m265) of a related strain S88, such that each recipient bacterium received a different plasmid member of the library. The mating procedures are routine and described in T. J. Pollock et al., Journal of Bacteriology, Volume 180, pages 586-593 (1998). Alternatively, one can routinely enrich for eps-negative mutants of Sphingomonas strain S7 or other Sphingomonas strains on agar plates containing YM and a growth-inhibiting concentration of bacitracin, for example 0.1 -10 mg/mL. Among the surviving bacitracin-resistant mutants of the parent strain will be a significant minority of eps-negative colonies which are recognizable because the colonies are translucent and watery compared to the opaque and rubbery eps+ parents. A small number of potential eps-negative isolates may be tested in shake flasks for absence of eps production, i.e., for the absence of viscosity in the broth or of isopropylalcohol-precipitable material. After the bacterial mating with the library, a few of the hundreds of recipient colonies that became Tet^{r} also exhibited synthesis of an exopolysaccharide as was evident by inspecting the colony appearances. Restoration of polysaccharide synthesis in the mutant by one of the cloned DNAs from the library caused that colony to be more opaque and rubbery in surface texture. The plasmids from several of the exopolysaccharide-positive colonies were isolated and analyzed for the specific pattern of cleavage by restriction endonucleases, and several unique segments of cloned DNA were recognized. One of these was clone S7c6 and it was compared to a previously cloned DNA segment from strain S88 for which the entire DNA sequence is known by DNA-DNA hybridization. The S7c6 clone contains gene sequences partially homologous to the spsGSRQKLJFDCEBrhsACBD cluster of genes from strain S88. A map of sites of cleavage for restriction endonucleases is shown in FIG. 1. A subclone of this cluster was prepared by digestion with restriction enzymes and contains only the spsBrhsACBD segment, which is abbreviated Brhs. Separately, the pRK311-S7c6 and pRK311-spsBrhsACBD plasmids were transferred by conjugation into the parental strain S7 for analysis of exopolysaccharide production.

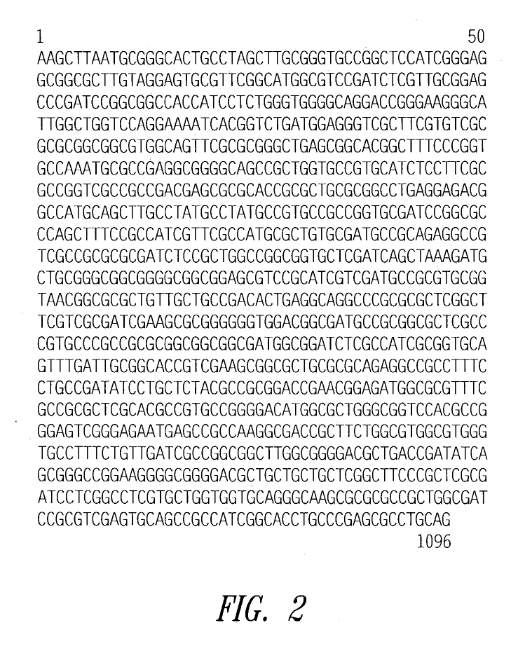

A segment of 1096 base pairs corresponding to the rightmost portion of the central 6.3 kbp BamHI-HindIII segment was sequenced. The DNA sequence from the S7c6 cloned segment is shown in FIG. 2. The sequence allows the construction of DNA-specific hybridization probes to screen libraries of segments from the chromosomal DNA. Thus, one does not need to use complementation of eps-negative mutants for the cloning of this S7 region.

Isolation of the Phosphoglucomutase Gene

A mutant of Escherichia coli (CGSC5527) deficient in phosphoglucomutase was obtained from the E. coli Genetic Stock Center (New Haven, Conn.), and used as a recipient for the entire S7 gene library. Of the hundreds of bacteria which received a plasmid, a few were restored to Pgm⁺. The Pgm⁺ exconjugants were observed as large white colonies on M63+galactose agar plates after over layering the colonies with iodine in dilute agar, while the parental Pgm⁻ mutants give black colonies. The screening method was described by Adhya and Schwartz, J. Bacteriol., Volume 108, page 621 (1971). The overlapping cloned pgm segments indicated that the region in common contained the pgm gene and this segment was cloned into the plasmid vector pRK311 and also into a small vector for DNA sequencing. The DNA sequence was determined and it showed considerable homology to other pgm genes isolated from other bacterial genera, eukaryotic microorganisms, plants and animals. The homology between the amino acid sequence of the Sphingomonas S7 pgm gene and the sequence of the Sphingomonas S60 gene (See Applied and Environmental Microbiology, Volume 66, pages 2252-2258 (2000)) is so extensive that both are expected to behave similarly when inserted into the Sphingomonas. Other related pgm genes are expected to also behave similarly in the context of our invention.

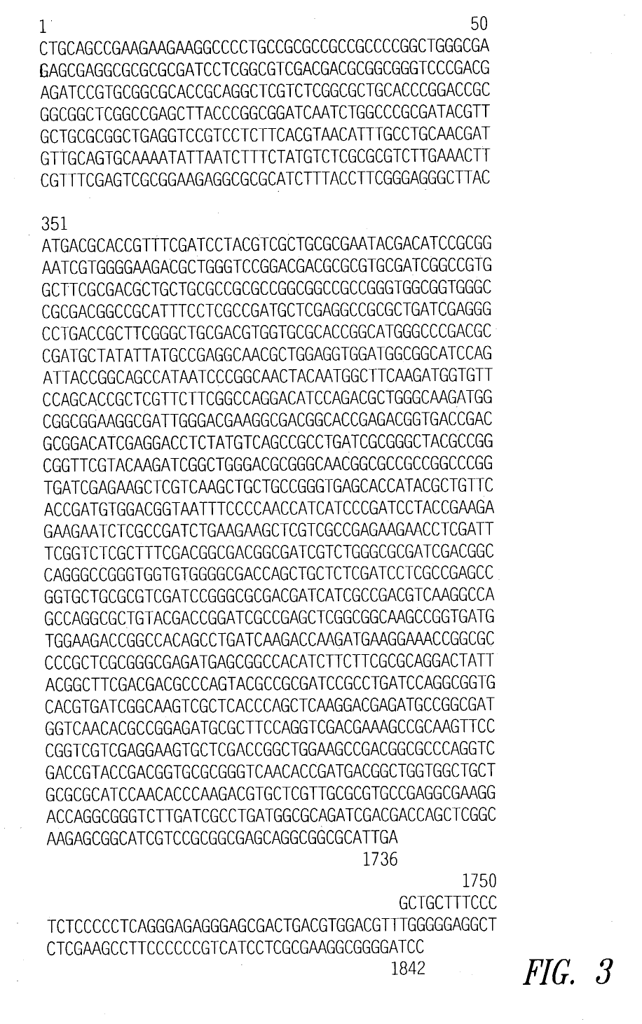

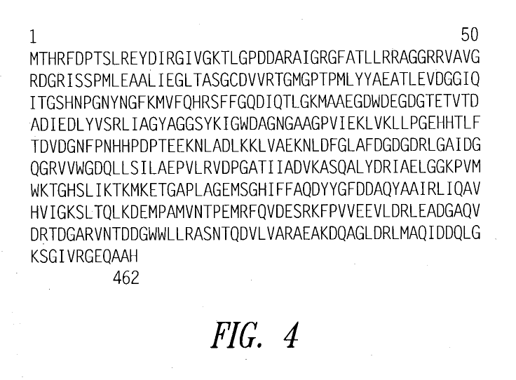

The DNA sequence is shown in FIG. 3 in which the bases which code for the amino acids of the PGM protein are between bases numbered 351 through 1736. The deduced amino acid sequence is shown in FIG. 4. The pgm gene was also cloned together with the spsBrhsACBD genes onto plasmid pRK311. Separately, the pRK311-pgm and pRK311-pgm-spsBrhsACBD plasmids, abbreviated as pRK-pgm and pRK-pgmBrhs, were transferred by conjugation into the parental strain S7 for analysis of exopolysaccharide production.

### Conversion Yields

The results of fermentations with unmodified and genetically modified derivatives of Sphingomonas strain S7 are shown below in Table 1.

TABLE 1

| | Absor- | | EPS | Residual | Conversion |
|---|---|---|---|---|---|
| Plasmid in | bance | Viscosity | S-7 | glucose | Yield (g EPS/g |
| strain S7 | 600 nm | (cp) | (g/l) | (g) | glucose) |
| none | 10.6 | 22500 | 16.5 | 0 | 52 |
| pRK-S7c6 | 7.8 | 30600 | 17.2 | 3 | 59 |
| pRK-Brhs | 9.3 | 20400 | 16.0 | 5 | 58 |
| pRK-pgm | 11.4 | 20900 | 16.0 | 3 | 55 |
| pRK-pgmBrhs | 9.1 | 23700 | 17.1 | 2 | 57 |

As readily apparent from Table 1, each of the modified strains converts a higher proportion of glucose into the product exopolysaccharide S-7. This indicates that multiple copies of genes isolated from the S7c6 sps gene cluster or of the pgm gene improve the productivity of strain S7. Either the entire S7c6 gene cluster can be used or a smaller segment including the spsB and rhsACBD genes.

As also shown in Table 1, the broth viscosity for strain S7 carrying additional copies of the plasmid pRK-S7c6 was increased compared to that of unmodified strain S7. After purification of the exopolysaccharide from the broth, the exopolysaccharide S7c6 (or “EPS S7c6”) retained its high viscosity as shown in Table 2. EPS S7c6 is the exopolysaccharide produced by the Sphingomonas strain S7 carrying plasmid pRK-S7c6. The increased viscosity per gram of purified exopolysaccharide suggested a new composition for the S7c6 polymer. The carbohydrate compositions for each of the exopolysaccharides from S7 and the genetically modified derivatives were determined following acid hydrolysis. The ratio of glucose to rhamnose is shown in Table 2 below. The EPS S7c6 has a unique sugar composition with relatively less glucose residues. Table 2 shows that the new composition is linked to the high viscosity.

The carbohydrate compositions were determined for samples of the culture broths after precipitation of the exopolysaccharide with 2 volumes of isopropyl alcohol. About 8-10 mg of dried material were hydrolyzed in 0.25 mL 2M trifluoroacetic acid at 100° C. for 4.5 hours, and then dried in a vacuum. The dry residue was resuspended in 0.05 mL of deionized water, dried again in a vacuum and finally resuspended in 0.2 mL pure water. The hydrolysate was passed through a spin filter and then 7.5 microliters were diluted with 493 microliters of pure water, and 10 microliters were applied to the chromatography column.

TABLE 2

| | | Carbohydrate |
|---|---|---|
| Source | | composition² |
| (cp) | Viscosity¹ | (glc:rha) |
| S7 | 2770 | 5.4 |
| unmodified | | |
| S7 with | 3810 | 4.2 |
| pRK-S7c6 | | |
| S7 with | 2270 | 5.6 |
| pRK-Brhs | | |
| S7 with | 2860 | 5.3 |
| pRK-pgm | | |

¹Measured with a Brookfield LVTDV-II viscometer with spindle 4 at 12 rpm and at 25° C.
²Given as the ratios of the peak areas on HPLC chromatograms for glucose and rhamnose

### Composition and Structure of the EPS S7c6

The structure of the extracellular polysaccharide S7c6 produced by Sphingomonas S7 containing plasmid pRK-S7c6 is composed of L-Rhap, D-Glcp, and 2-deoxy-β-D-arabino-HexpA (hereinafter referred as “2-deoxy-HexpA”) in the molar ratios 1:3:1 (2-deoxy-HexpA is 2-deoxyglucuronic acid). The 2-deoxy-HexpA residue is acid-labile and was not detected by glycosyl residue and glycosyl-linkage composition analyses. Its presence was established by ¹H and ¹³C NMR spectroscopy which also established the relative amounts of the glycosyl constituents. EPS S7c6 is partially fragmented by β-elimination upon treatment with NaOH and deuterium-labeled methyl iodide (C₂H₃I). The fragments thus formed consist of a series of per-O-trideuteriomethylated oligosaccharides each of which is terminated at their non-reducing end with a Δ-4,5-2-deoxy-HexpA residue. Glycosyl linkage composition analysis, MALDI-TOF-MS, and one- and two dimensional-¹H and ¹³C NMR spectroscopy of these oligosaccharides established that EPS S7c6 is composed predominantly of the following pentasaccharide repeating unit:

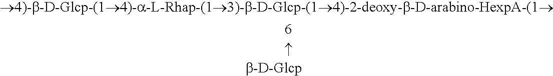

The terminal β-D-Glcp residue is absent in ˜10% of the repeating units, while another ˜10% of the repeating units have a second β-D-Glcp- attached to O-6 of what was the terminal β-D-Glcp residue →. Thus, the repeating unit of EPS S7c6 can be unsubstituted or substituted with a mono- or diglucosyl side chain. The length of the side chains is the only detectable difference between EPS S7c6 and EPS S-7 which is the polysaccharide synthesized by the parent bacterium. Each repeating unit of EPS S-7 has a diglucosyl side chain.

### Experimental Procedures Used in Determining the Composition and Structure of EPS S7c6

Glycosyl-Residue Composition Analysis

The neutral glycosyl- composition of the neutral glycosyl residues of EPS S7c6 was determined by GC analysis of the alditol acetate derivatives. The polysaccharide (500 μg) was treated for 1 hour at 121° C. with 2 M trifluoroacetic acid (200 μL) containing myo-inositol (20 μg) as an internal standard. The acid was evaporated under a flow of nitrogen gas and the residue was washed with methanol (2×500 μL). The residue was dissolved in water and the resulting solution treated for 1 hour at room temperature with NaBD₄ (10 mg/mL in M NH₄OH) to convert the released monosaccharides to their corresponding alditols. The alditols were then acetylated by treatment for 20 min at 121° C. with pyridine (100 μL) and acetic anhydride (100 μL). The reagents were removed by codistillation with toluene and the residue was suspended in water (1 mL). Chloroform (1 mL) was added and the organic phase containing the acetylated alditols concentrated to dryness. The residue was dissolved in acetone (100 μL) and a portion (1 μL) analyzed by GC-MS using a 30 m SP 2330 capillary column and a HP 580 Mass Selective Detector (MSD).

The neutral and acidic glycosyl residue composition of the polysaccharide was determined by GC analysis of the trimethylsilyl (TMS) methyl glycoside derivatives. The polysaccharide (550 μg) was treated for 16 hours at 80° C. with anhydrous methanol containing 1 M HCI (250 μL) and myo-inositol (20 μg). The methanol was removed under a flow of nitrogen gas and the residue washed with isopropanol (3×500 μL). A solution of the residue in Tri-Sil (Pierce, 100 μL) was kept for 20 min at 80° C. to convert the methyl glycosides to their corresponding TMS derivatives. The reagents were removed under a flow of nitrogen gas and the residue dissolved in hexane (100 μL). A portion (1 μL) of the solution was then analyzed by GC-MS using a 30 m DB-1 capillary column and a HP 5890 MSD.

Glycosyl-Linkage Composition Analysis

A solution of the polysaccharide (1 μg) in dimethylsulfoxide (250 μL) was methylated using solid NaOH and iodomethane (Ciucanu and Kerek, Carbohydr Res 131, 209-217(1984)). The reaction was quenched by the addition of water (1 mL) and the per-O-methylated polysaccharide extracted into chloroform (1 mL). The organic phase was concentrated to dryness and then treated for 1 hour at 121° C. with 2 M trifluoroacetic acid (200 μL) containing myo-inositol (20 μg). The acid was removed under a flow of nitrogen gas and the residue washed with methanol (2×500 μL). A solution of the residue was then dissolved in and reacted for 1 hour at room temperature with NaBD₄ (10 mg/mL in M NH₄OH) to convert the released partially methylated methyl glycosides to their corresponding alditols. The partially methylated alditols were acetylated by treatment for 3 hours at 121° C. with acetic anhydride (100 μL). Excess acetic anhydride was removed by co-distillation with toluene and the residue suspended in water (1 mL). Chloroform (1 mL) was added and the organic phase containing the methylated alditol acetates concentrated to dryness. The residue was dissolved in acetone (100 μL) and a portion (1 μL) was analyzed by GC-MS using a 30 m SP 2330 capillary column and a HP 580 MSD.

Preparation of the Trideuteriomethylated Polysaccharide

A solution of the polysaccharide (30 mg) in dimethylsulfoxide (2 mL) was methylated using solid NaOH and deuteriomethyl iodide (Ciucanu and Kerek, Carbohydr Res 131, 209-217(1984)). The reaction was quenched by the addition of water (2 mL) and the per-O-deuteriomethylated polysaccharide extracted into chloroform (1 mL). The organic phase was concentrated to dryness and then dissolved to deuterated chloroform. (500 μL).

¹H and ¹³C Nuclear Magnetic Resonance Spectroscopy

¹H and ¹³C NMR spectroscopy were performed with a Varian Inova 800 MHz NMR spectrometer at 25° C. Gradient-selected COSY, TOCSY, and HSQC experiments were performed using the pulse sequence programs supplied by the manufacturer.

Matrix-Assisted Laser-Desorption Time-of-Flight Mass Spectrometry

Matrix-assisted laser-desorption time-of-flight mass spectrometry (MALDI-TOF-MS) was performed with a Hewlett Packard (Cupertino, Calif.) LDI 1700XP mass spectrometer operated at 30 kV accelerating voltage in the positive ion mode. A portion (1 μL) of a solution of the per-O-deuteriomethylated polysaccharide(100 μg) in methanol (100 μL) was mixed with a solution (1 μL) of dihydroxybenzonic acid (DHB, 5 mg/mL in acetonitrile) and applied to the surface of the MS probe. The probe was then placed under vacuum to remove the organic solvents and co-crystallize the trideuteriomethylated material with the DHB matrix.

Glycosyl-Residue and Glycosyl-Linkage Compositions

EPS S7c6 was shown by glycosyl-residue composition analysis to contain Rha and Glc in the molar ratio 1.0:2.6 (See the glucosyl residue composition of EPS S7c6 in Table 3 below). No hexuronic acid residues were detected by analysis of the TMS methyl glycoside derivatives (See Table 3). Table 3 shows the mole % of rhamnosyl and glucosyl in alditol acetate and TMS methyl glycoside.

TABLE 3

| Glycosyl residue | Alditol acetate | TMS methyl glycoside |
|---|---|---|
| Rhamnosyl | 28 | 32 |
| Glucosyl | 72 | 68 |

Glycosyl-linkage composition analysis showed that the polysaccharide contains terminal non-reducing Glcp, 4-Glcp, 6-Glcp, 3-Glcp, 3,6-Glcp, and 4-linked Rhap in the molar ratios 1.0:2.5:0.7:0.6:1.0:2.7 (See the glycosyl-linkage composition of EPS S7c6 in Table 4 below).

TABLE 4

| Glycosyl Linkage | Molar Ratio |
|---|---|
| T-Glcp | 1.0 |
| 3-Glcp | 0.6 |
| 6-Glcp | 0.7 |
| 4-Glcp | 2.5 |
| 3,6-Glcp | 1.0 |
| 4-Rhap | 2.7 |

These results when taken together with the glycosyl sequence of EPS S-7 suggest that EPS S7c6 is composed predominantly of the following pentasaccharide repeating unit:

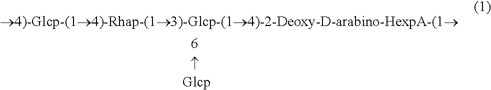

The presence of 3-linked Glcp (See Table 4) can be accounted for by the absence of the T-Glcp side chain in about 10% of the repeating units (See Structure 2 below), whereas the presence of 6-linked Glcp (See Table 4) is likely to result from the presence, in about 10% of the repeating units, of a diglucosyl side chain linked to the backbone (See Structure 3 below).

→4)-Glcp-(1→4)-Rhap-(1→3)-Glcp-(1→4)-2-Deoxy-β-D-arabino-HexpA-(1→ (2)

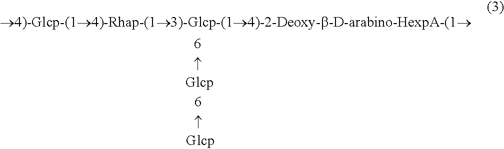

MALDI-TOF-MS of the Per-O-Trideuteriomethylated Polysaccharide

The results of glycosyl-residue and glycosyl-linkage composition analyses of EPS S7c6 together with glycosyl sequence of EPS S-7 indicates that the polysaccharide from the mutant is composed predominantly of a pentasaccharide repeating unit composed of three Glc residues, one Rha residue and one 2-deoxy-β-D-arabino-HexpA. Additional information about the glycosyl sequence of EPS S7c6 was obtained by MALDI-TOF-MS and NMR spectroscopic analyses of the products generated by per-O-deuteriomethylation of EPS S7c6.

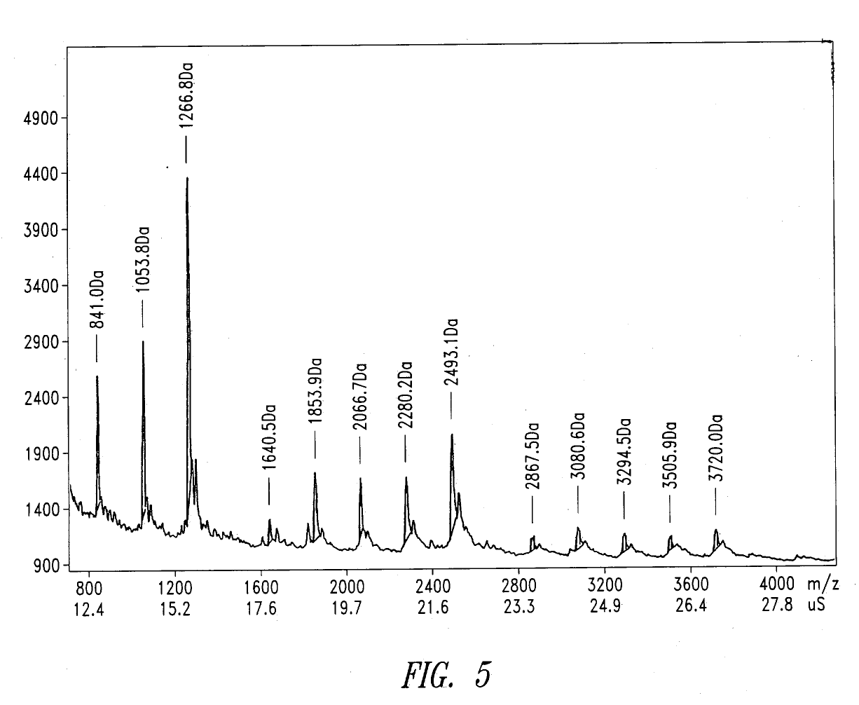

The MALDI-TOF mass spectrum of per-O-deuteriomethylated EPS S7c6 (See FIG. 5) provides strong evidence that the polysaccharide had indeed been fragmented during alkylation. Moreover, the MS analysis is consistent with the presence of a 2-deoxy-HexpA residue in addition to the Rha and Glc residues.

The MALDI-TOF mass spectrum of the per-O-deuteriomethylated polysaccharide (See FIG. 5) contains signals between m/z 800 and 4000 that correspond to the [M+Na]⁺ ions of a series of per-O-deuteriomethylated oligosaccharides (See the MALDI-TOF-MS analysis of the products generated by per-O-deuteriomethylation of EPS S7c6 in Table 5 below).

TABLE 5

| [M + Na]⁺ | Deduced glycosyl residue composition of | | | | |
|---|---|---|---|---|---|
| ion³ | EPS S7c6 fragment⁴ | | | | |
| Measured | Calculated | Glu- | | 2-deoxy- | 2-deoxy- |
| Mass | Mass | cosyl | Rhamnosyl | HexA | HexA-4,5 |
| 841 | 841 | 2 | 1 | 0 | 1 |
| 1054 | 1054 | 3 | 1 | 0 | 1 |
| 1267 | 1267 | 4 | 1 | 0 | 1 |
| 1641 | 1641 | 4 | 2 | 1 | 1 |
| 1854 | 1854 | 5 | 2 | 1 | 1 |
| 2067 | 2068 | 6 | 2 | 1 | 1 |
| 2280 | 2281 | 7 | 2 | 1 | 1 |
| 2493 | 2494 | 8 | 2 | 1 | 1 |
| 2868 | 2868 | 8 | 3 | 2 | 1 |
| 3081 | 3081 | 9 | 3 | 2 | 1 |
| 3295 | 3294 | 10 | 3 | 2 | 1 |
| 3506 | 3507 | 11 | 3 | 2 | 1 |
| 3720 | 3720 | 12 | 3 | 2 | 1 |

³MALDI-TOF spectra were obtained in the positive ion mode
⁴The glycosyl residue composition determined from the [M + Na]⁺ ion.

The mass of the oligosaccharide obtained by MS can be seen in FIG. 5. The difference in the measured and expected masses is due to non-linearities in the MS calibration at high mass range.

The intensities of the ions do not reflect the amount of a particular oligosaccharide that is present since MALDI-TOF-MS is not quantitative. The ions at m/z 841, 1054 and 1267 correspond to the [M+Na]⁺ ions of oligosaccharide derivatives composed of one Rha residue, one unsaturated 2-deoxy-HexpA residue, and one, two, or three Glc residues, respectively. These results taken together with the glycosyl linkage composition of EPS S7c6 (See Table 4) are consistent with the presence of a tetrasaccharide (See Structure 4 below), a pentasaccharide (See Structure 5 below), and a hexasaccharide (See Structure 6 below).

Δ4:5dioxyHexA-4Glc-4Rha-3Glc-OCD₃ (4)

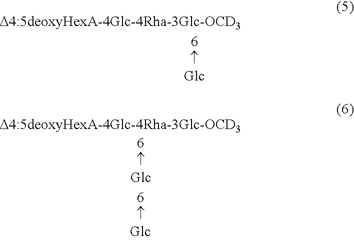

The series of ions between m/z 1600 and 2500 correspond to [M+Na]⁺ ions of oligosaccharide derivatives composed of between 8 and 12 glycosyl residues. These oligosaccharides contain a Δ-4,5-2-deoxy-HexpA residue at the terminal non-reducing end internally as well as an internal 4-linked 2-deoxy-β-D-arabino-HexpA residue. The ions at m/z 1854 and 2280 are likely to correspond to Structure 7 below and Structure 8 below, respectively.

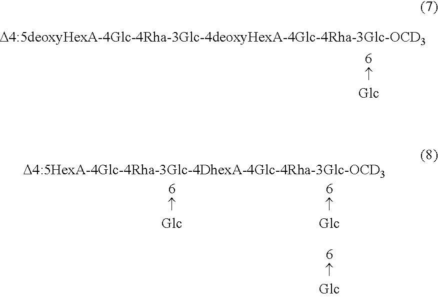

The ion at m/z 2067 may correspond to either Structure 9 below or Structure 10 below since both derivatives have the same molecular mass.

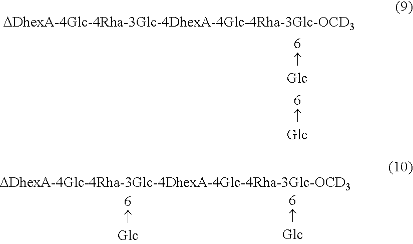

These results indicate that EPS S7c6 is a single polysaccharide with variable length side chains (0,1, or 2 glycosyl residues on each repeat). The results tend to rule out the possibility that EPS S7c6 is a mixture of three different polysaccharides with the same backbone, that is, where one polysaccharide has no side chains, a second polysaccharide has a single glycosyl-residue side chain on each repeat, and the third has a two glucosyl-residue side chain on each repeat.

¹H and ¹³C NMR Spectroscopy of the Per-O-Deuteriomethylated Polysaccharide

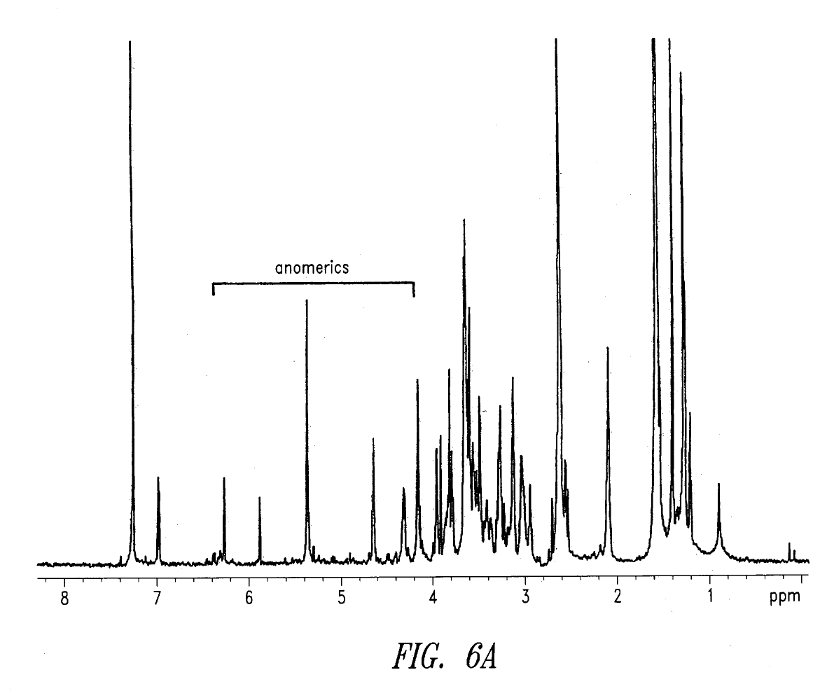

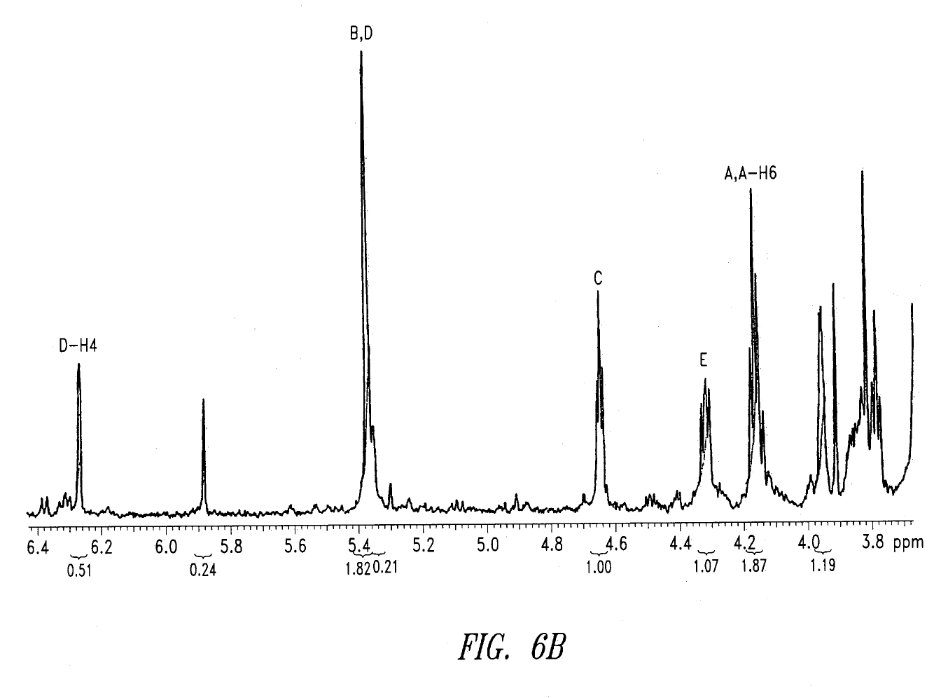

The 1D ¹H spectra of per-O-deuteriomethylated EPS S7c6 and EPS S-7 are similar, although the EPS S7c6 spectrum is more complex (See FIG. 6A). Both spectra contain sharp signals that are typical of low molecular weight oligosaccharides. Thus, EPS S7c6 and EPS S-7 are both partially fragmented during per-O-trideuteriomethylation. The ¹H NMR spectrum of EPS S7c6 (See FIG. 6A and Table 6 below which shows the assignment of the signals in the ¹H NMR spectrum of per-O-deuteriomethylated EPS S7c6) is consistent with the presence of a pentasaccharide (as shown as Structure 5 above and again below) terminated at the non-reducing end by a HexpA-4,5-ene residue (hereinafter “D”) and with the methyl glycoside of 3,6-linked glycosyl residue (hereinafter “A”) at the reducing terminus. Residues A and D are the expected derivatives of base-catalyzed β-elimination at C4 of the 4-linked 2-deoxy-β-D-arabino-HexpA residue.

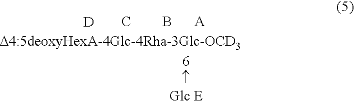

TABLE 6

| Residue | H1 | H2 | H3 | H4 | H5 | H6 | H6 |
|---|---|---|---|---|---|---|---|
| A 3,6-Glc | 4.171 | 3.014 | 3.65 | 3.05 | 3.416 | 4.16 | 3.65 |
| B 4-Rha | 5.37 | 3.608 | 3.51 | 3.66 | 3.84 | 1.27 | — |
| C 4-Glc | 4.645 | 2.946 | 3.127 | 3.792 | 3.275 | 3.65 | 3.49 |
| D T-2- | 5.368 | 2.099 | 3.961 | 6.273 | — | — | — |
| deoxy- | | | | | | | |
| HexpA-4,5- | | | | | | | |
| ene | | | | | | | |
| E T-Glc | 4.313 | 3.034 | 3.13 | 3.11 | 3.26 | 3.63 | 3.57 |

In Table 6, the chemical shifts are in ppm from TMS and measured from internal CDCI₃ set to δ7.27.

The hexasaccharide generated by per-O-deuteriomethylation of EPS S-7 (See Structure 6) and Structure 5 have identical backbones. However, most of the polysaccharides in EPS S7c6 lack the β-D-glucosyl residue linked to O6 of residue E from Structure 5 above. The ¹H NMR spectrum of EPS S7c6 is consistent with the presence of ˜15% 4-linked 2-deoxy-β-D-arabino-HexpA residues, which are present in those oligosaccharides that contain more than one repeating unit.

The anomeric region (δ 4.0-6.4) of the ¹H NMR spectrum of per-O-deuteriomethylated EPS S7c6 contains more signals than would be expected from a homogeneous polysaccharide composed of a pentasaccharide repeating unit (See FIG. 6B). Indeed, the ¹H NMR spectrum of deuteriomethylated EPS S7c6 is consistent with the presence of small amounts of Structure 6, which contains a β-D-glucosyl residue linked to O-6 of residue E from Structure 5 above. However, this could not be unambiguously confirmed from the NMR data due to sample heterogeneity and signal overlap, although it was confirmed by MALDI-TOF mass spectrometry (See above).

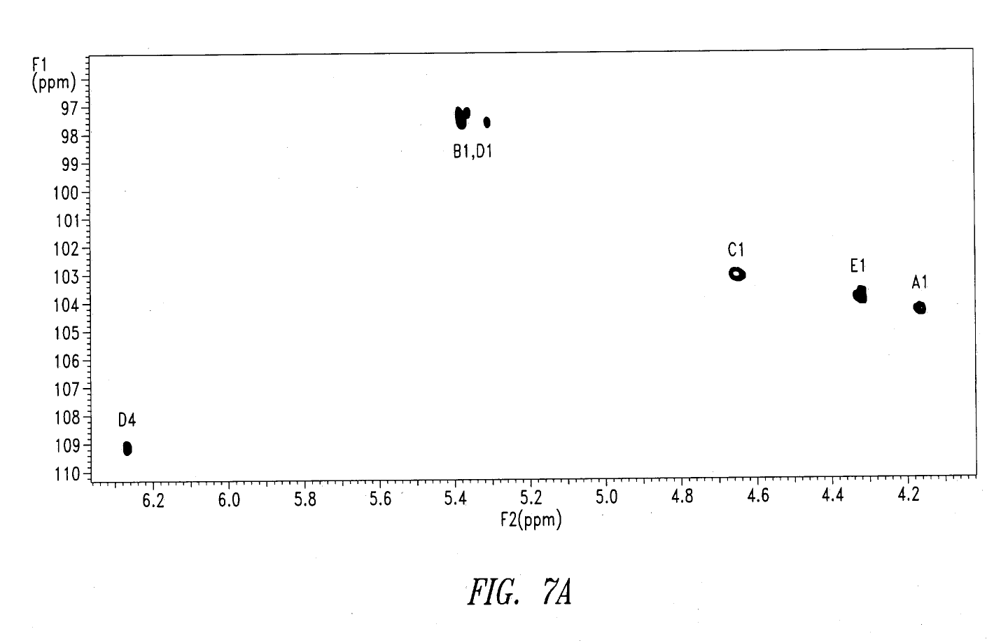

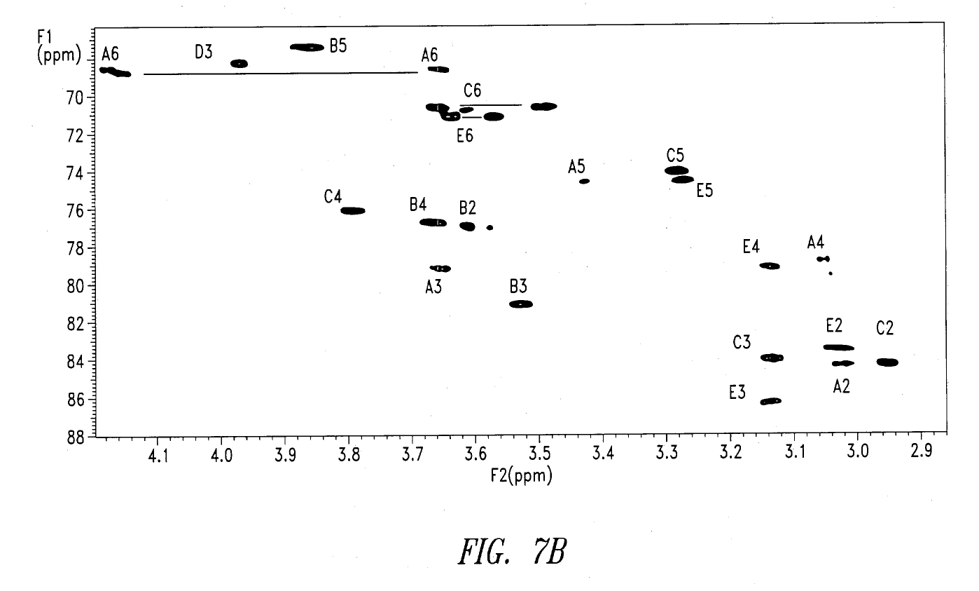

The positions of the glycosidic linkages of Structure 5 and their anomeric configurations were determined using the 2D NMR experiments known as COSY, TOCSY and HSQC (See FIGS. 7A and 7B). The results of these experiments taken together with those of 1D ¹³C NMR spectroscopy (See Table 7 below which shows the assignment of the signals in the ¹³H NMR spectrum of per-O-deuteriomethylated EPS S7c6) and the previously obtained assignments of EPS S-7 provide the structure of Structure 5.

TABLE 7

| Residue | C1 | C2 | C3 | C4 | C5 | C6 |
|---|---|---|---|---|---|---|
| A 3,6-Glc | 104.4 | 84.52 | 79.32 | 78.94 | 74.71 | 68.6 |
| B 4-Rha | 97.6 | 77.05 | 81.18 | 76.83 | 67.4 | 18.3 |
| C 4-Glc | 103.1 | 84.43 | 84.18 | 76.18 | 74.17 | 70.8 |
| D T-2- | 97.4 | 33.3 | 68.3 | 109.1 | — | — |
| deoxy- | | | | | | |
| HexpA-4,5- | | | | | | |
| ene | | | | | | |
| E T-Glc | 103.9 | 83.65 | 86.41 | 79.30 | 74.63 | 71.3 |

In Table 7, the chemical shifts are in ppm from TMS and measured from internal CDCI₃ set to δ77.23.

The glycosidic linkages were confirmed by the results of a 2D NOESY NMR experiment. The results of these experiments also confirmed that per-O-deuteriomethylation of EPS S7c6 generates a mixture of structurally related oligosaccharides. For example, the 1D ¹H spectrum of Structure 5 contains quantitatively minor glycosyl residue signals indicative of the presence of residues in amounts less than one in five residues (See FIGS. 6A and 6B). Furthermore, integration of the anomeric signals indicated that they are present in amounts that are less than one in five residues. These results taken together with those of MALDI-TOF-MS provide strong evidence that EPS S7c6 is fragmented by the strong base used to catalyze per-O-methylation of EPS S7c6.

The signal at δ6.27 in the 1D ¹H spectrum of Structure 5 is assigned to H-4 of the 4,5-unsaturated 2-deoxy-β-D-arabino-HexpA (residue D in Structure 5). 2D COSY, TOCSY and HSQC experiments established that the signal at ˜δ5.37 corresponds to H-1 of the Δ-4,5-2-deoxy-HexpA derivative and to H-1 of the rhamnosyl residue (residue B in Structure 5), and that the signal at δ2.099 corresponds to H-2 of the Δ-4,5-2-deoxy-HexpA residue (See Table 6). The assignments for H-1 of the 4-rhamnosyl, 4-glucosyl, 3,6-glucosyl, and terminal-glucosyl residues (See Table 6) are consistent with the previous assignments of the ¹H NMR spectrum of the per-O-deuteriomethylated EPS S-7.

A NOESY spectrum of per-O-deuteriomethylated EPS S7c6 shows strong NOEs between H-1 of Δ-4,5-2-deoxy-HexpA residue D and H-4 of glucosyl residue C in Structure 5 above and between H-1 of glucosyl residue C and H-4 of rhamnosyl residue B in Structure 5. Weak NOEs are present between H-1 of rhamnosyl residue B and H-3 of glucosyl residue A in Structure 5. Other weak signals in the spectrum are compatible with NOEs between H-1 of the terminal glucosyl residue E and H-6 of glucosyl residue A in Structure 5.

EPS S7c6 and EPS S-7 both contain a 3,6-linked glucosyl residue and a terminal non-reducing glucosyl residue. EPS S-7 also contains a 6-linked glucosyl residue that has characteristic ¹H resonances at δ4.31 for H-1, and δ4.14 and δ3.63 for the H-6s. The COSY spectrum of EPS S7c6 contains a portion of the spin system H-6/H-6′-H-5 -H-4 that was assigned to the 6-linked glucosyl in EPS S-7. Moreover, the ¹H -¹³C HSQC spectrum of EPS S7c6 (See FIG. 7B) shows that the signals assigned to C6-H6 of the 3,6-glucosyl residue contain shoulders that may result from the presence of a 6-linked glucosyl residue.

The results of this study have established that the polysaccharide secreted by the Sphingomonas S7 containing plasmid pRK-S7c6 is composed predominantly of the pentasaccharide repeat unit (Structure 1). EPS S-7, the polysaccharide synthesized by the parent bacterium, is composed of a hexasaccharide repeating unit. The 6-linked Glcp residue in Structure 1 above is β in both EPS S7c6 and EPS S-7 whereas it is α in the hexasaccharide repeating unit of the S. paucimobillis polysaccharide characterized by Falk et al (1996). These are the only three bacterial polysaccharides known to contain a 2-deoxy-β-D-arabino-HexpA residue. These polysaccharides are likely to contain O-acetyl groups and may also contain phosphate esters. However, the locations of these putative non-carbohydrate substituents have not been determined.

The 2-deoxy-β-D-arabino-HexpA residue degrades when EPS S7c6 or EPS S-7 is treated with hot acid and thus is not detected by glycosyl-residue and glycosyl-linkage composition analyses. The glycosidic bond between the 3,6-linked Glcp residue and the 2-deoxy-HexpA residue is partially cleaved by base-catalyzed β-elimination when EPS S7c6 or EPS S-7 is alkylated with NaOH/C₂H₃I. This results in the partial fragmentation of the polysaccharide and the generation of a series of alkylated oligosaccharides that are terminated at their non-reducing end with a 4,5-unsaturated derivative of a 2-deoxy-arabino-HexpA residue. The glycosyl sequences of the EPS S7c6 fragments were determined using NMR spectroscopy, MALDI-TOF-MS and glycosol-linkage composition analysis. The results obtained from these experiments in conjunction with those obtained during characterization of EPS S-7 define the chemical structure of the repeating unit of EPS S7c6, the polysaccharide secreted by Sphingomonas S7 containing plasmid pRK-S7c6.

### DEPOSITS

The following two bacterial strains were deposited with the Patent Depository at the American Type Culture Collection at 10801 University Boulevard, Manassas, Va. 20110, on Jun. 29, 2000 pursuant to the Budapest Treaty for the International Recognition of the Deposit of Microrganisms:

(1) Sphingomonas strain S7 with plasmid pRK311-S7c6, also denoted as S7/pRK-S7c6; and

(2) Sphingomonas strain S7 with plasmid pRK311-pgm spsB rhsACBD, also denoted as S7/pRK-pgmBrhs.

What is claimed is:

1. A polysaccharide produced by subjecting a Sphingomonas bacterium modified with a S7c6 gene cluster or segment thereof to aerobic fermentation in a nutrient aqueous broth for a time sufficient to produce the polysaccharide dissolved therein, the polysaccharide comprising L-Rhap, D-Glcp and 2-deoxy-β-D-arabino-HexpA in a molar ratio of 1:3:1, wherein the polysaccharide has at least 20% less glucose per repeat unit compared to a heteropolysaccharide S-7 produced by an unmodified Sphingomonas strain S7, and the segment comprises at least the spsB and rhsACBD genes.

2. A fermentation broth containing the polysaccharide of claim 1.

3. The polysaccharide of claim 1 having at least 25% less glucose per repeat unit compared to the heteropolysaccharide S-7.

4. A per-O-deuteriomethylated polysaccharide produced by subjecting the polysaccharide of claim 1 to methylation, the per-O-deuteriomethylated polysaccharide having the matrix-assisted laser-desorption time-of-flight (MALDI-TOF) mass spectrum of FIG. 5.

5. A per-O-deuteriomethylated polysaccharide produced by subj ecting the polysaccharide of claim 1 to methylation, the per-O-deuteriomethylated polysaccharide having the nuclear magnetic resonance (NMR) spectrum of FIG. 6A.

6. A per-O-deuteriomethylated polysaccharide produced by subjecting the polysaccharide of claim 1 to methylation, the per-O-deuteriomethylated polysaccharide having the ¹H -¹³C HSQC spectrum (anomeric region) of FIG. 7A.

7. A per-O-deuteriomethylated polysaccharide produced by subjecting the polysaccharide of claim 1 to methylation, the per-O-deuteriomethylated polysaccharide having the ¹H -¹³C HSQC spectrum (ring carbons) of FIG. 7B.

8. A method for increasing the viscosity of an aqueous solution comprising adding to the aqueous solution a viscosity increasing effective amount of a polysaccharide comprising L-Rhap, D-Glcp and 2-deoxy-β-D-arabino-HexpA in a molar ratio of 1:3:1, wherein the polysaccharide is produced by subjecting a Sphingomonas bacterium modified with a S7c6 gene cluster or segment thereof to aerobic fermentation in a nutrient aqueous broth for a time sufficient to produce the polysaccharide dissolved therein, and the segment comprises at least the spsB and rhsACBD genes.

9. The method of claim 8 wherein the polysaccharide has at least 20% less glucose per repeat unit compared to a heteropolysaccharide S-7 produced by an unmodified Sphingomonas strain S7.

10. The method of claim 9 wherein the polysaccharide has at least 25% less glucose per repeat unit compared to the heteropolysaccharide S-7.

11. A fermentation broth obtained by subjecting a Sphingomonas bacterium modified with a S7c6 gene cluster or segment thereof to aerobic fermentation in a nutrient aqueous broth for a time sufficient to produce a dissolved polysaccharide, wherein the polysaccharide comprises L-Rhap, D-Glcp and 2-deoxy-β-D-arabino-HexpA in a molar ratio of 1:3:1, the polysaccharide has at least 20% less glucose per repeat unit compared to a heteropolysaccharide S-7 produced by an unmodified Sphingomonas strain S7, and the segment comprises at least the spsB and rhsACBD genes.

12. The fermentation broth of claim 11 wherein the polysaccharide has at least 25% less glucose per repeat unit compared to the heteropolysaccharide S-7.


SEQUENCE LISTING (3 sequences)

SEQ ID NO: 1 - DNA, 1096 bp, Sphingomonas sp.
aagcttaatgcgggcactgcctagcttgcgggtgccggctccatcgggaggcggcgcttgtaggagtgcgttcggcatggcgtccgatctcgttgcggagcccgatccggcggccaccatcctctgggtggggcaggaccgggaagggcattggctggtccaggaaaatcacggtctgatggagggtcgcttcgtgtcgcgcgcggcggcgtggcagttcgcgcgggctgagcggcacggctttcccggtgccaaatgcgccgaggcggggcagccgctggtgccgtgcatctccttcgcgccggtcgccgccgacgagcgcgcaccgcgctgcgcggcctgaggagacggccatgcagcttgcctatgcctatgccgtgccgccggtgcgatccggcgcccagctttccgccatcgttcgccatgcgctgtgcgatgccgcagaggccgtcgccgcgcgcgatctccgctggccggcggtgctcgatcagctaaagatgctgcgggcggcggggcggcggagcgtccgcatcgtcgatgccgcgtgcggtaacggcgcgctgttgctgccgacactgaggcaggcccgcgcgctcggcttcgtcgcgatcgaagcgcggggggtggacggcgatgccgcggcgctcgcccgtgcccgccgcgcggcggcggcgatggcggatctcgccatcgcggtgcagtttgattgcggcaccgtcgaagcggcgctgcgcgcagaggccgcctttcctgccgatatcctgctctacgccgcggaccgaacggagatggcgcgtttcgccgcgctcgcacgccgtgccggggacatggcgctgggcggtccacgccgggagtcgggagaatgagccgccaaggcgaccgcttctggcgtggcgtgggtgcctttctgttgatcgccggcggcttggcggggacgctgaccgatatcagcgggccggaaggggcggggacgctgctgctgctcggcttcccgctcgcgatcctcggcctcgtgctggtggtgcagggcaagcgcgcgccgctggcgatccgcgtcgagtgcagccgccatcggcacctgcccgagcgcctgcag

SEQ ID NO: 2 - DNA, 1842 bp, Sphingomonas sp.
ctgcagccgaagaagaaggcccctgccgcgccgccgccccggctgggcgagagcgaggcgcgcgcgatcctcggcgtcgacgacgcggcgggtcccgacgagatccgtgcggcgcaccgcaggctcgtctcggcgctgcacccggaccgcggcggctcggccgagcttacccggcggatcaatctggcccgcgatacgttgctgcgcggctgaggtccgtcctcttcacgtaacatttgcctgcaacgatgttgcagtgcaaaatattaatctttctatgtctcgcgcgtcttgaaacttcgtttcgagtcgcggaagaggcgcgcatctttaccttcgggagggcttacatgacgcaccgtttcgatcctacgtcgctgcgcgaatacgacatccgcggaatcgtggggaagacgctgggtccggacgacgcgcgtgcgatcggccgtggcttcgcgacgctgctgcgccgcgccggcggccgccgggtggcggtgggccgcgacggccgcatttcctcgccgatgctcgaggccgcgctgatcgagggcctgaccgcttcgggctgcgacgtggtgcgcaccggcatgggcccgacgccgatgctatattatgccgaggcaacgctggaggtggatggcggcatccagattaccggcagccataatcccggcaactacaatggcttcaagatggtgttccagcaccgctcgttcttcggccaggacatccagacgctgggcaagatggcggcggaaggcgattgggacgaaggcgacggcaccgagacggtgaccgacgcggacatcgaggacctctatgtcagccgcctgatcgcgggctacgccggcggttcgtacaagatcggctgggacgcgggcaacggcgccgccggcccggtgatcgagaagctcgtcaagctgctgccgggtgagcaccatacgctgttcaccgatgtggacggtaatttccccaaccatcatcccgatcctaccgaagagaagaatctcgccgatctgaagaagctcgtcgccgagaagaacctcgatttcggtctcgctttcgacggcgacggcgatcgtctgggcgcgatcgacggccagggccgggtggtgtggggcgaccagctgctctcgatcctcgccgagccggtgctgcgcgtcgatccgggcgcgacgatcatcgccgacgtcaaggccagccaggcgctgtacgaccggatcgccgagctcggcggcaagccggtgatgtggaagaccggccacagcctgatcaagaccaagatgaaggaaaccggcgccccgctcgcgggcgagatgagcggccacatcttcttcgcgcaggactattacggcttcgacgacgcccagtacgccgcgatccgcctgatccaggcggtgcacgtgatcggcaagtcgctcacccagctcaaggacgagatgccggcgatggtcaacacgccggagatgcgcttccaggtcgacgaaagccgcaagttcccggtcgtcgaggaagtgctcgaccggctggaagccgacggcgcccaggtcgaccgtaccgacggtgcgcgggtcaacaccgatgacggctggtggctgctgcgcgcatccaacacccaagacgtgctcgttgcgcgtgccgaggcgaaggaccaggcgggtcttgatcgcctgatggcgcagatcgacgaccagctcggcaagagcggcatcgtccgcggcgagcaggcggcgcattgagctgctttccctctccccctcagggagagggagcgactgacgtggacgtttgggggaggctctcgaagccttccccccgtcatcctcgcgaaggcggggatcc

SEQ ID NO: 3 - protein, 462 aa, Sphingomonas sp.
MTHRFDPTSLREYDIRGIVGKTLGPDDARAIGRGFATLLRRAGGRRVAVGRDGRISSPMLEAALIEGLTASGCDVVRTGMGPTPMLYYAEATLEVDGGIQITGSHNPGNYNGFKMVFQHRSFFGQDIQTLGKMAAEGDWDEGDGTETVTDADIEDLYVSRLIAGYAGGSYKIGWDAGNGAAGPVIEKLVKLLPGEHHTLFTDVDGNFPNHHPDPTEEKNLADLKKLVAEKNLDFGLAFDGDGDRLGAIDGQGRVVWGDQLLSILAEPVLRVDPGATIIADVKASQALYDRIAELGGKPVMWKTGHSLIKTKMKETGAPLAGEMSGHIFFAQDYYGFDDAQYAAIRLIQAVHVIGKSLTQLKDEMPAMVNTPEMRFQVDESRKFPVVEEVLDRLEADGAQVDRTDGARVNTDDGWWLLRASNTQDVLVARAEAKDQAGLDRLMAQIDDQLGKSGIVRGEQAAH